(19) United States
(12) Patent Application Publication
(10) Pub. No.: US 2022/0064687 A1
(43) Pub. Date: Mar. 3, 2022

(54) HYDROGEL COMPOSITION, MANUFACTURING METHOD THEREOF AND ENZYMATICALLY FORMED HYDROGEL COMPOSITION

(71) Applicant: BIOGEND THERAPEUTICS CO., LTD., Taipei City (TW)

(72) Inventors: Er-Yuan CHUANG, Taipei City (TW); Po-Wei LEE, Taipei City (TW); Po-Yen LIN, Taipei City (TW)

(21) Appl. No.: 17/462,849

(22) Filed: Aug. 31, 2021

Related U.S. Application Data
(60) Provisional application No. 63/072,484, filed on Aug. 31, 2020.

(51) Int. Cl. C12P 19/04; C08J 3/075; C08J 3/24

(52) U.S. Cl. CPC C12P 19/04; C08J 3/075; C12Y 114/18001; C12Y 111/01007; C08J 3/24

(57) ABSTRACT

Disclosed are a hydrogel composition, a method for manufacturing the hydrogel composition, and an enzymatically formed hydrogel composition, all of which are characterized mainly by the use of calcium peroxide as an oxygen receptor in a tyrosinase-catalyzed polymer crosslinking reaction in order for the polymer to chelate with calcium ions. The resulting hydrogel composition not only is highly biocompatible, but also has desirable mechanical properties and a high gelation speed.

### BACKGROUND OF THE INVENTION

### 1. Technical Field

The present invention relates mainly to but is not limited to the field of chemical compositions. More particularly, the invention relates to a hydrogel composition, a method for manufacturing the same, and an enzymatically formed hydrogel composition.

### 2. Description of Related Art

For years, articular cartilage tissue has continued to be a subject of great importance in fields related to tissue regeneration. Cartilage tissue has a complicated biological structure, relatively low metabolic activity, and no blood vessels and is therefore difficult, if not impossible, to regenerate and repair. Cartilage tissue also has quite a limited self-repairing ability such that surgical treatment is often required. A tiny crack in an articular cartilage may gradually enlarge and result in osteoarthritis if not treated in time, even though it is less than 2 cm long and has no symptom at all in the first place.

Currently, methods commonly used to repair small defects in cartilage tissue include, for example, mosaicplasty and microfracture surgery. These methods, however, tend to give rise to the development of fibrocartilage rather than hyaline cartilage and do not provide the desired biological integrability with the neighboring native organs. As a solution, synthetic biocompatible materials are gradually adopted as substitutes for use in cartilage repair. One highly notable example of such biomaterials is hydrogel compositions.

Hydrogel compositions can be used to construct gelled biopolymers such as hyaluronic acid, alginate, and chitosan. As is well known in the art, gelled biopolymers can be formed in many ways. Some injectable or implantable polymeric gel systems can be directly delivered to a lesion in a minimally invasive manner and gel in situ and are therefore highly valued.

### BRIEF SUMMARY OF THE INVENTION

The inventor of the present invention has found that articular cartilage tissue is a physiological environment with a low oxygen concentration, which may inhibit the oxidation reactions of enzymes. Moreover, some polymeric materials exhibit relatively low adhesion due to a lack of cell-binding peptides. It is therefore an important issue in the technical field to which the present invention pertains to enable a biomaterial that gels in vivo and in situ to adhere to the surrounding tissue and have mechanical strength and biocompatibility at the same time.

In view of the aforesaid issues of the prior art, the present invention provides a novel concept as detailed below. After validating that a calcium ion can chelate with the carboxyl group in an alginate polymer, the inventor of the present invention has found that calcium peroxide can serve as an oxygen receptor in, and thereby enhance, a tyrosinase-catalyzed polymer crosslinking reaction, and that the calcium ion provided by calcium peroxide can further chelate with the polymer to form a hydrogel composition that has high biocompatibility, high adhesion, and a high gelation speed.

More specifically, a first aspect of the present invention relates to a hydrogel composition comprising a plurality of polymers, wherein each of the polymers includes a backbone, and the backbone includes a plurality of carboxyl groups and a branch formed by tyramine; wherein any two of the polymers have a bond between adjacent branches, and at least one of the plurality of carboxyl groups is chelated with a calcium ion.

According to an embodiment of the present invention, the backbone is selected from the group consisting of gelatin, chitosan, heparin, cellulose, dextran, dextran sulfate, chondroitin sulfate, keratan sulfate, dermatan sulfate, alginate, collagen, albumin, fibronectin, laminin, elastin, vitronectin, hyaluronic acid, fibrinogen, a multi-arm polymer and a combination thereof.

According to an embodiment of the present invention, the backbone is a polysaccharide copolymer or a polysaccharide homopolymer.

According to an embodiment of the present invention, the bond is an enzymatic oxidative coupling.

Preferably, the enzymatic oxidative coupling is formed by in situ cross-linking of the polymers in an environment with an enzyme and calcium peroxide, wherein the concentration of calcium peroxide is between 0.4 and 1 mM, and the enzyme is tyrosinase or horseradish peroxidase.

Another aspect of the present invention relates to an enzymatically formed hydrogel composition as represented by formula (II), which includes: a plurality of polymers, each of which comprises a homogenous or heterogeneous backbone with a structure as represented by formula (I); wherein at least one carboxyl group of the backbone and at least one calcium ion are chelated into a structure of formula (II),

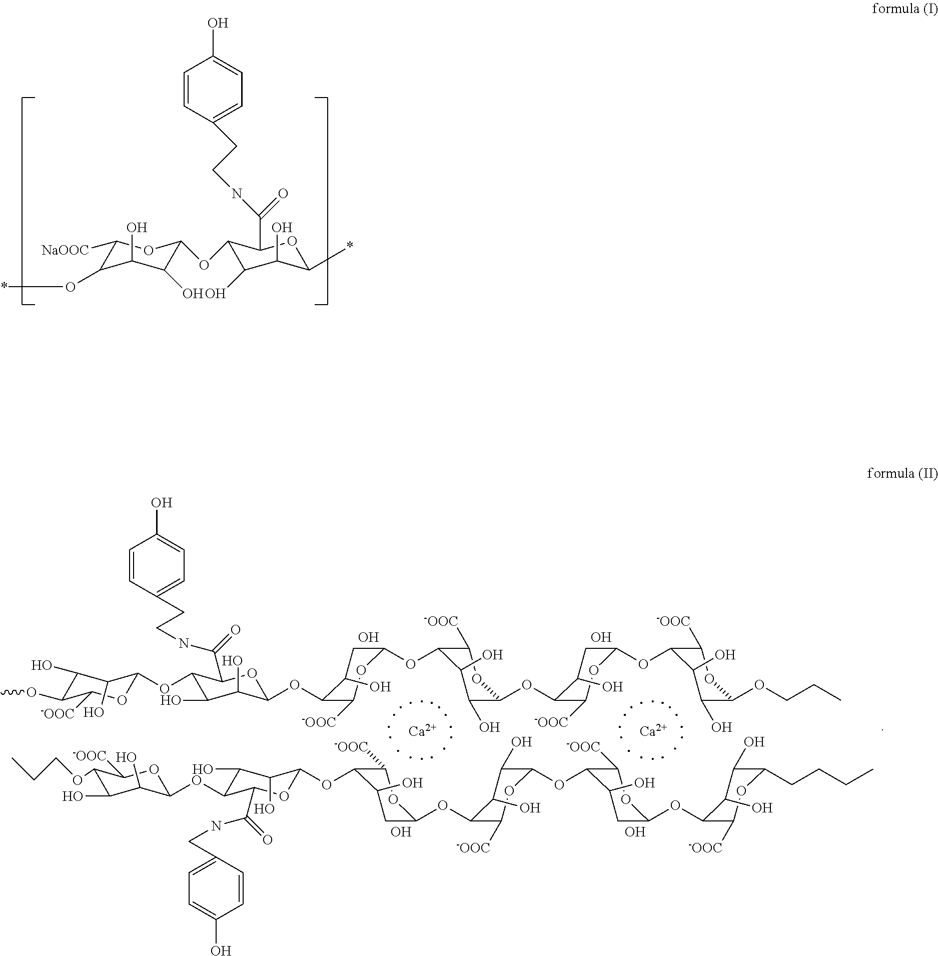

According to an embodiment of the present invention, the backbone includes a branch formed by tyramine, and adjacent branches on any two of the polymers have a bond as shown in the structure of formula (III):

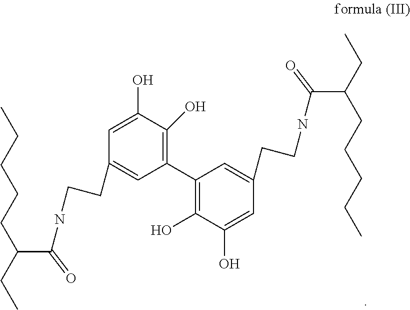 [SMILES: CCCCCC(CC)C(=O)NCCc1cc(O)c(O)c(-c2cc(CCNC(=O)C(CC)CCCCC)cc(O)c2O)c1]

According to an embodiment of the present invention, wherein the structure of formula (II) and the bond of the structure of formula (III) are formed by in-situ cross-linking of the polymers in an environment with an oxidase and calcium peroxide.

Preferably, the oxidase is tyrosinase or horseradish peroxidase.

Preferably, the concentration of calcium peroxide is between 0.4 and 1mM.

Another aspect of the present invention relates to a method for manufacturing a hydrogel composition, which includes the following steps:
(a) reacting tyramine with a backbone of a polymer to produce a precursor polymer; and
(b) adding a cross-linking accelerator and an ion chelating agent to cross-link a plurality of the precursor polymers to form a hydrogel composition.

Preferably, in step (a), the reaction ratio of the polymer and tyramine is between 1:0.4 and 1:6.

Preferably, the cross-linking accelerator is tyrosinase or horseradish peroxidase.

Preferably, the ion chelating agent is calcium peroxide.

Preferably, in step (b), the reaction concentration of the ion chelating agent is between 0.4 and 1 mM.

The present invention is advantageous over the prior art in that the hydrogel composition disclosed herein, the method disclosed herein for manufacturing the hydrogel composition, and the enzymatically formed hydrogel composition disclosed herein provide not only high biocompatibility, but also high adhesion and a high gelation speed.

The summary of the invention aims to provide a simplified summary of the disclosure, so that the reader has a basic understanding of the disclosure. This summary of the invention is not a complete overview of the disclosure, and it is not intended to point out important/critical elements of embodiments of the invention or define the scope of the invention.

### BRIEF DESCRIPTION OF THE SEVERAL VIEWS OF THE DRAWINGS

In order to make the above and other objects, features, advantages and embodiments of the present invention more obvious and understandable, the drawings are described as follows:

FIG. 1(a) to (d) schematically show the chemical reactions involved in an embodiment of the present invention;

FIG. 2 shows ¹H NMR spectra corresponding to an embodiment of the invention;

FIG. 3 shows FTIR spectra corresponding to the same embodiment as FIG.

2;

FIG. 4 shows gelation times corresponding to an embodiment of the invention;

FIG. 5 shows adhesive stress measurements corresponding to the same embodiment as FIG. 4;

FIG. 6A is a plot showing an MTT cell survival rate analysis result corresponding to an embodiment of the invention, and FIG. 6B shows a LIVE/DEAD cell viability assay result corresponding to the same embodiment;

FIG. 7 shows gelation times corresponding to an embodiment of the invention;

FIG. 8 shows FTIR spectra corresponding to an embodiment of the invention;

FIG. 9 shows SEM images corresponding to an embodiment of the invention;

FIG. 10A is a plot showing a stress-time relationship corresponding to an embodiment of the invention, and FIG. 10B is a plot showing a stress-distance relationship corresponding to the same embodiment;

FIG. 11 shows rheological property test results corresponding to the same embodiment as FIG. 10A and FIG. 10B;

FIG. 12 shows optical microscope images corresponding to an embodiment of the invention;

FIG. 13A shows immunohistochemical analysis results of the experimental group and control group in an embodiment of the invention, FIG. 13B shows more immunohistochemical analysis results of the experimental group in the same embodiment, and FIG. 13C shows immunohistochemical analysis results of certain soft tissues adjacent to the implanted experimental group/control group in the same embodiment;

FIG. 14A shows images of the surgical operations performed on, and of the appearances of, the portions implanted with the experimental group or control group in an embodiment of the invention, and FIG. 14B shows immunohistochemical analysis results of the experimental group and control group in the same embodiment;

FIG. 15A and FIG. 15B are plots showing hydrogel adhesion analysis results and overall hydrogel tensile strength analysis results corresponding to an embodiment of the invention;

FIG. 16 shows optical/fluorescence microscope images corresponding the same embodiment as FIG. 15A and FIG. 15B;

FIG. 17 is a plot showing an MTT cell viability assay result corresponding to the same embodiment as FIG. 15A and FIG. 15B; and

FIG. 18 is a plot showing a stress-strain relationship corresponding to an embodiment of the invention.

According to the usual working methods, the various features and components in the figures are not drawn according to the actual scale. The drawings present the specific features and components related to the present invention in the best way. In addition, between different drawings, the same or similar element symbols refer to similar elements and components.

### DETAILED DESCRIPTION OF THE INVENTION

In this section, the contents of the present invention will be described in detail through the following examples. These examples are for illustration only, and those skilled in the art can easily think of various modifications and changes. Various embodiments of the present invention will be described in detail below. In this specification and the appended patent applications, unless the context clearly indicates otherwise, “a” and “the” can also be interpreted as plural. In addition, in this specification and the scope of the attached patent application, unless otherwise stated in the context, “middle” and “inner” include “located in”; and unless otherwise stated in the context, the direction of the tip of the projectile was defined as “upper” or “lower”. Furthermore, titles and subtitles may be attached to the description for easy reading, but these titles do not affect the scope of the present invention.

Although the numerical ranges and parameters used to define the present invention are approximate values, the relevant values in the specific embodiments have been presented as accurately as possible. However, any numerical value inevitably contains standard deviations due to individual test methods. Here, “about” generally means that the actual value is within plus or minus 10%, 5%, 1%, or 0.5% of a specific value or range. Or, the term “about” means that the actual value falls within the acceptable standard error of the average value, which is determined by those with ordinary knowledge in the field to which the present invention belongs. Therefore, unless otherwise stated to the contrary, the numerical parameters disclosed in this specification and the accompanying patent application are approximate values and can be changed as required. At least these numerical parameters should be understood as the indicated significant digits and the values obtained by applying the general rounding method.

One objective of the present invention is to provide a hydrogel composition comprising a plurality of polymers, wherein each of the polymers includes a backbone, and the backbone includes a plurality of carboxyl groups and a branch formed by tyramine; wherein any two of the polymers have a bond between adjacent branches, and at least one of the plurality of carboxyl groups is chelated with a calcium ion.

Another objective of the present invention is to provide an enzymatically formed hydrogel composition as represented by formula (II), which includes: a plurality of polymers, each of which comprises a homogenous or heterogeneous backbone with a structure as represented by formula (I); wherein at least one carboxyl group of the backbone and at least one calcium ion are chelated into a structure of formula (II),

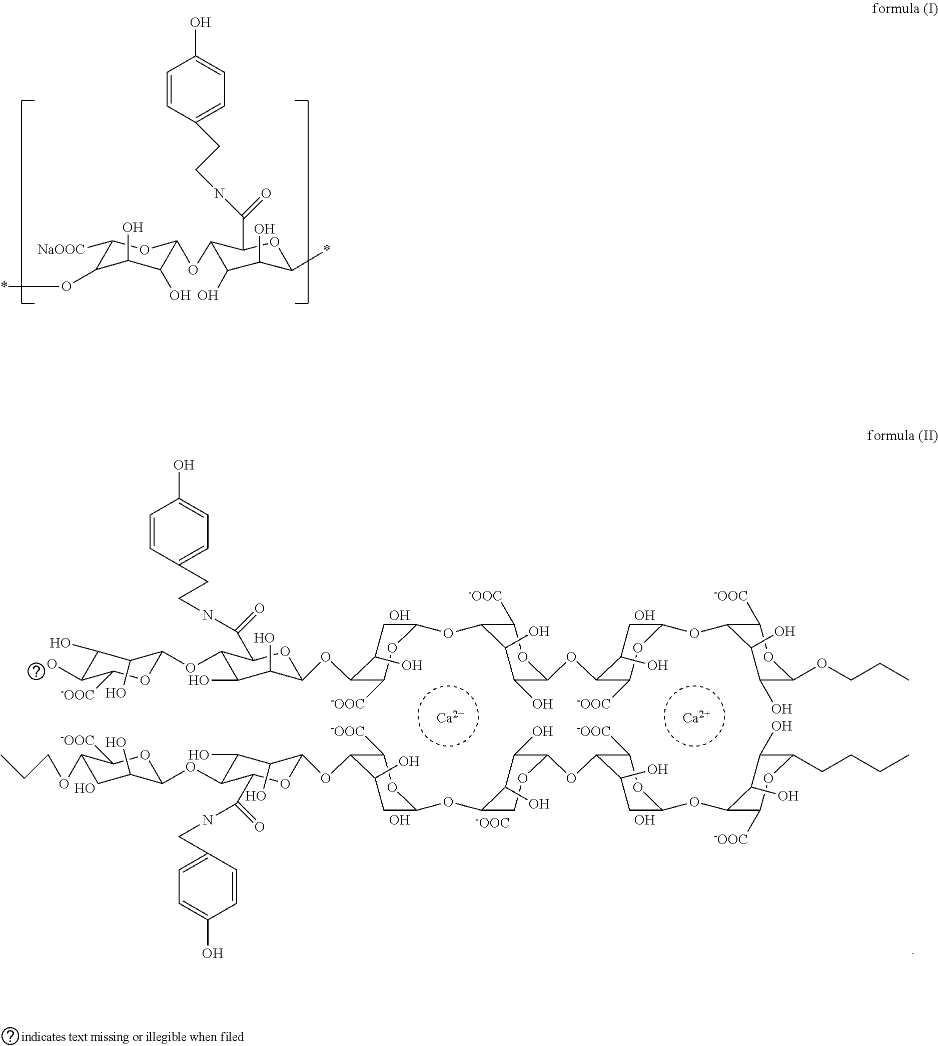

As used herein, the term “hydrogel composition” refers to a hydrophilic polymer having a three-dimensional web-like structure formed by a crosslinking reaction of molecular chains such that after absorbing water, the polymer can expand without disintegration. Any water-soluble or hydrophilic polymers having a functional group such as —OH, —CONH, —CONH₂, or —COOH can form a hydrogel composition by either chemical or physical crosslinking. The aforesaid polymers can be generally divided into natural ones, synthetic ones, and a combination thereof. Natural hydrophilic polymers include polysaccharides (e.g., cellulose, starch, hyaluronic acid, alginic acid, and chitosan) and polypeptides (e.g., collagen, poly-L-lysine, and poly-L-glutamic acid). Synthetic hydrophilic polymers include alcohols, acrylic acid, and derivatives thereof, such as polyacrylic acid, polymethacrylic acid, and polyacrylamide. In some embodiments of the present invention, each of the polymers involved in forming a hydrogel composition has a backbone selected from the group consisting of gelatin, chitosan, heparin, cellulose, dextran, dextran sulfate, chondroitin sulfate, keratan sulfate, dermatan sulfate, alginate, collagen, albumin, fibronectin, laminin, elastin, vitronectin, hyaluronic acid, fibrinogen, a multi-arm polymer, and a combination thereof.

In addition/alternatively, the aforesaid backbone in some embodiments of the present invention may be a polysaccharide copolymer or a polysaccharide homopolymer. In some embodiments, the aforesaid backbone is a polysaccharide copolymer formed by copolymerization of mannuronate and guluronate. In some embodiments, the aforesaid backbone is a polysaccharide homopolymer formed by polymerization of mannuronate and guluronate.

As used herein, the term “enzyme-catalyzed oxidative coupling” refers generally to the bond, typically a covalent bond, formed between two molecules or functional groups by an enzyme-catalyzed oxidation reaction. More specifically, in some embodiments of the present invention, the term refers to the crosslinking bond formed between molecular chains of polymers by an enzyme-catalyzed oxidation reaction. In some embodiments of the invention, this bond is an enzyme-catalyzed oxidative coupling. In some embodiments, the bond is an enzyme-catalyzed oxidative coupling including an intermediate product, and the intermediate product is o-benzoquinone.

In some embodiments, the bond between the adjacent braches of any two polymers involved in forming a hydrogel composition is an amide bond. In some embodiments of the present invention, the backbone of each polymer involved in forming a hydrogel composition includes a branch formed by tyramine, and the bond between the adjacent braches of any two such polymers has the structure of formula (III):

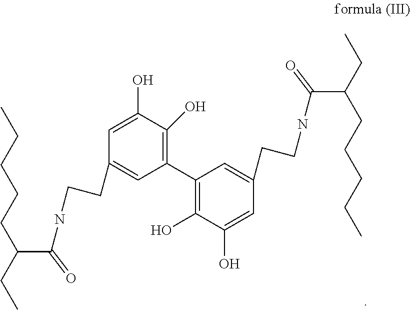 [SMILES: CCCCCC(CC)C(=O)NCCc1cc(O)c(O)c(-c2cc(CCNC(=O)C(CC)CCCCC)cc(O)c2O)c1]

As used herein, the term “in situ crosslinking” refers to the crosslinking of polymers in a particular physiological environment, initiated by changing a physical property or chemical parameter (e.g., temperature, pH value, or ion concentration) of the polymers. More specifically, in some embodiments of the present invention, a physical property or chemical parameter of the polymers in a hydrogel composition injected into a target tissue is controlled in order for the polymers to crosslink in situ, thereby turning the hydrogel composition from a liquid or sol state into a gel state. In some embodiments of the invention, an enzyme-catalyzed oxidative coupling is formed by in situ crosslinking of a plurality of polymers in an environment where an enzyme and calcium peroxide are present, with the concentration of the calcium peroxide being between 0.4 and 1 mM, and the enzyme being tyrosinase or horseradish peroxidase. In some other embodiments of the invention, the structure of formula (II) and a bond of the structure of formula (III) are formed by in situ crosslinking of polymers in an environment where an oxidase and calcium peroxide are present. More specifically, the oxidase is tyrosinase or horseradish peroxidase, and tyrosinase is used in the following embodiments. In addition, the concentration of the calcium peroxide is between 0.4 and 1 mM, preferably between 0.4 and 0.8 mM. The concentration of the calcium peroxide may be, but is not limited to, 0.4, 0.5, 0.6, 0.7, 0.8, 0.9, 1 mM, or any value therebetween.

Another objective of the present invention is to provide a method for manufacturing a hydrogel composition, which includes the following steps:
(a) reacting tyramine with a backbone of a polymer to produce a precursor polymer; and
(b) adding a cross-linking accelerator and an ion chelating agent to cross-link a plurality of the precursor polymers to form a hydrogel composition.

In some embodiments of the present invention, the reaction ratio of the polymer to the tyramine in step (a) is between 1:0.4 and 1:6, preferably between 1:0.4 and 1:5. For example, the reaction ratio of the polymer to the tyramine is 1:0.4, 1:0.5, 1:0.6, 1:0.7, 1:0.8, 1:0.9, 1:1, 1:1.1, 1:1.2, 1:1.3, 1:1.4, 1:1.5, 1:1.6, 1:1.7, 1:1.8, 1:1.9, 1:2.1, 1:2.3, 1: 1:2.5, 1:2.7, 1:2.9, 1:3.1, 1:3.3, 1:3.5, 1:3.7, 1:3.9, 1:4.1, 1:4.3, 1:4.5, 1:4.7, 1:4.9, 1:5.1, 1:5.3, 1:5.5, 1:5.7, 1:5.9 or 1:6.

In some embodiments of the present invention, the crosslinking accelerator is tyrosinase or horseradish peroxidase. In a preferred embodiment, the crosslinking accelerator is tyrosinase.

In some embodiments of the present invention, the reaction concentration of the ion chelating agent in step (b) is between 0.4 and 1 mM, preferably between 0.4 and 0.8 mM. For example, the concentration of the ion chelating agent is 0.4, 0.5, 0.6, 0.7, 0.8, 0.9, 1 mM, or any value therebetween. In some embodiments of the invention, the ion chelating agent is calcium peroxide. Without being limited by any theory, the inventor of the invention has found that if the ion chelating agent is calcium peroxide, the calcium ion of the calcium peroxide will produce a chelating effect in the hydrogel composition such that gaps are formed in the hydrogel composition, meaning there will still be space in the hydrogel composition after the hydrogel composition solidifies. When the hydrogel composition is put to actual use, therefore, an exchange of gas can take place, or tissue fluid can be discharged, through the hydrogel composition, thereby increasing the survival rate of cells covered with the hydrogel composition.

### Embodiments

Overview of the Reactions Involved

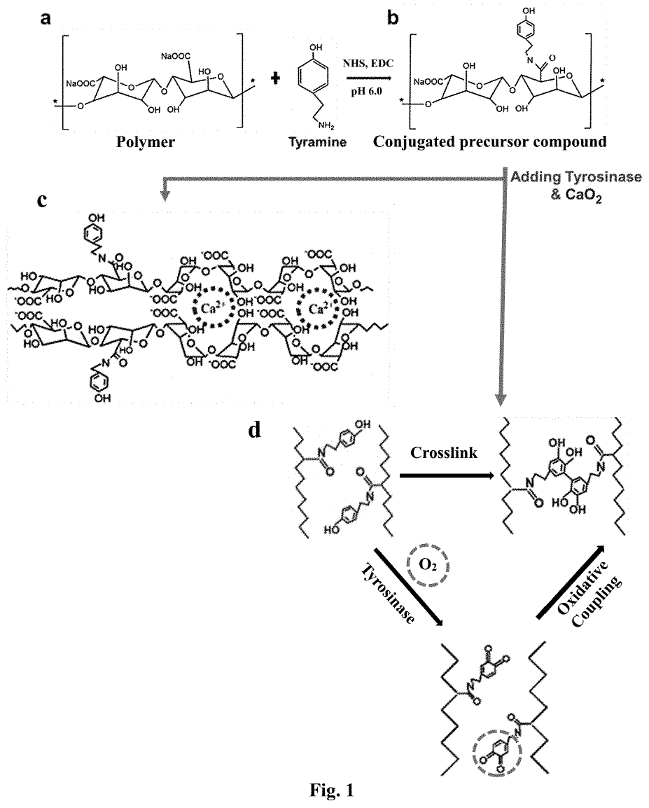

FIG. 1 schematically shows the chemical reactions involved in an embodiment of the present invention and illustrates the basic chemical reactions involved in the hydrogel composition disclosed herein, in the method disclosed herein for manufacturing the hydrogel composition, and in the enzymatically formed hydrogel composition disclosed herein. Please note that the reference characters (a)-(d) in FIG. 1 are used to indicate the process flow of the chemical reactions only approximately; (c) and (d) occur substantially at the same time. As shown in FIG. 1, the first step is to react a polymer with tyramine (hereinafter referred to as TYR for short) so as to produce a conjugated precursor compound. More specifically, the polymer is an alginate (hereinafter referred to as ALG for short), or sodium alginate to be exact. After that, tyrosinase and calcium peroxide are added to the conjugated precursor compound, in order for the tyrosinase to catalyze oxidation of the adjacent phenolic groups in the conjugated precursor compound into quinonyl groups, with the calcium peroxide acting as an oxygen receptor. The quinonyl groups will couple with each other such that crosslinking and gelation take place (as indicated by the reference character (d)). In the meantime, the calcium peroxide provides a calcium ion, which chelates with at least one of the plurality of carboxyl groups of the conjugated precursor compound to form an egg-box-shaped crosslinked structure (as indicated by the reference character (c)). The following paragraphs are based on the chemical reactions stated above and detail the experiment processes and results of some embodiments of the invention.

1. Preparation of a Precursor Compound from ALG and TYR

Sodium alginate is dissolved in deionized water (DI H₂O) (2 wt %) and then mixed with the same amount of MES (2-(N-morpholino)ethanesulfonic acid) buffer solution (0.5 M). After that, the ALG (i.e., sodium alginate) is pretreated by reacting the carboxyl group of the ALG with an activator containing water-soluble carbodiimide in order to form an amide bond. In this embodiment, the activator containing water-soluble carbodiimide includes a 1-ethyl-3-(3-dimethylaminopropyl)carbodiimide compound (EDC) and an N-hydroxysuccinimide compound (NHS). The reaction molar ratio of the ALG, NHS, and EDC in the pretreatment is 1:1.5:1.5. In addition, a helium-based degassing step is performed to prevent redox reactions with oxygen, and HCl or NaOH is added to adjust the pH value to 5.0 and thereby enhance the reaction of the pretreatment. After the pretreatment reaction continues for 30 minutes, the pretreated ALG is reacted with TYR to produce an ALG-TYR conjugated precursor compound. More specifically, the pretreated ALG is reacted with the TYR at different ratios, and the reactions and their products are divided by the ratio into different groups. The reaction molar ratios of the pretreated ALG and the TYR are 1:0.4, 1:1, 1:2.5, 1:5, and 1:6, which correspond to groups R0.4, R1, R2.5, R5, and R6 respectively. The pretreated ALG and the TYR in each group are mixed into an MES buffer solution, and the pH value of the mixture is adjusted to about 5.5 to accelerate the reaction so that after 24 hours, an ALG-TYR precursor compound corresponding to a particular reaction molar ratio is obtained.

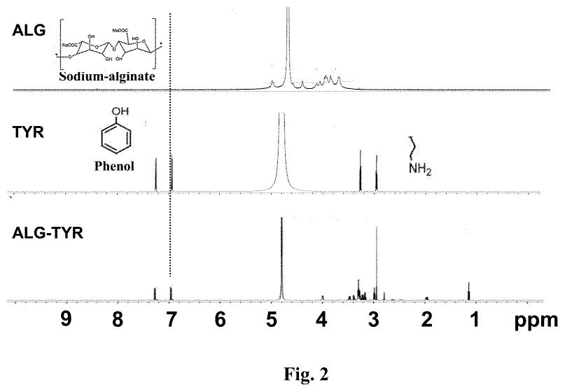

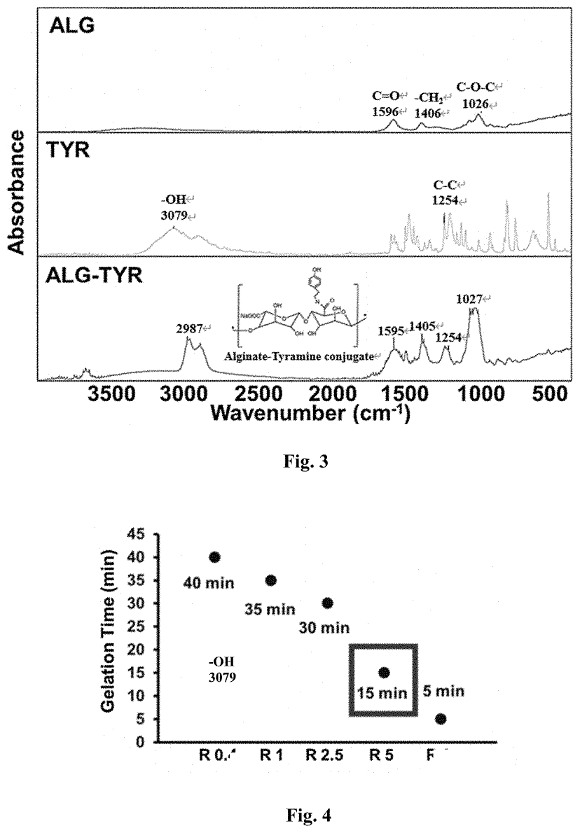

The following step of this embodiment is to identify the chemical structures, and verify the grafted states, of the ALG-TYR precursor compounds obtained. More specifically, the identification is carried out by nuclear magnetic resonance (NMR) and Fourier-transform infrared spectroscopy (FTIR). The main working principle of NMR is to detect the radio-frequency signals released by a spinning atomic nucleus when the nucleus is excited into a high-energy state by an applied magnetic field and then returns to the equilibrium state. The detected signals allow the structure, dynamic state, and so on of a molecule to be observed. FTIR involves detecting the specific infrared energy absorbed by a sample and generating a spectrum accordingly. The resulting spectrum can be used to identify the functional groups of a molecule. Referring to FIG. 2 for ¹H NMR spectra corresponding to this embodiment, the ALG spectrum shows the typical homopolymeric and heteropolymeric block fractions of an alginate composed of mannuronate or guluronate (at about 4-5 ppm), and the ALG-TYR precursor compound spectrum shows the aromatic protons of the phenolic group of the TYR (at about 7.0 ppm). Referring to FIG. 3 for FTIR spectra corresponding to this embodiment, the characteristic peaks of the ALG are located at 1596, 1406, and 1026 cm⁻¹ and correspond to C═O, CH₂, and —C—O—C— respectively, and the characteristic peaks of the TYR are located at 3079 and 1254 cm⁻¹ and correspond to the hydroxyl group and the C—C functional group respectively. A comparison of the spectra in FIG. 3 indicates that the TYR has been grafted onto the ALG, and the actual grafted state of the ALG-TYR precursor compound under test is thus verified.

This embodiment also determines the degrees of substitution (D.S.) of the ALG-TYR precursor compounds with a spectrometer. As used herein, the term “degree of substitution” refers to the average number of conjugated substituents per base unit or per monomeric unit in a polymeric material. In some embodiments of the present invention, the degree of substitution refers to the average number of conjugated TYR per base unit or per monomeric unit in an ALG-TYR precursor compound. Accurate assessment of the D.S. value, however, is no easy task. In this embodiment, therefore, the D.S. percentages of the precursor compounds in groups R0.4, R1, R2.5, R5, and R6 are determined by measuring the 275-nm absorbance of the TYR, and the results are shown in Table 1, in which it can be seen that the D.S. increases with the molar ratio of the TYR in a reaction.

TABLE 1

| | ALG | TYR | | OD 275 | |
|---|---|---|---|---|---|
| Groups | (mmol) | (mmol) | Ratio | (nm) | D.S. (%) |
| R0.4 | 2.5 | 1 | 1:0.4 | 0.2538 | 16.49% |
| R1 | 2.5 | 2.5 | 1:1 | 0.2945 | 18.49% |
| R2.5 | 2.5 | 6.25 | 1:2.5 | 0.342 | 22.24% |
| R5 | 2.5 | 12.5 | 1:5 | 0.378 | 27.89% |
| R6 | 2.5 | 15 | 1:6 | 0.46 | 32.48% |

2. Addition of Tyrosinase to Cause Gelation of the ALG-TYR Precursor Compound

To find the technical features of the present invention that can bring about relatively good results, experiments and analyses are performed on variables related to the properties of the enzymatically gelled products in this embodiment.

2-1 Degree of Substitution (D.S.)

2-1-1 Analysis of Mechanical Properties Corresponding to Different D.S. Values

Generally, the D.S. value of a polymeric material is in direct proportion to the mechanical strength, in particular tensile strength, of a product made of the polymeric material. To validate this observation, this embodiment is carried out by mixing the precursor compound in each of groups R0.4, R1, R2.5, R5, and R6 (in which the D.S. value varies from one group to another) with tyrosinase of a fixed concentration of 10 KU to form a to-be-tested hydrogel composition (hereinafter referred to as a to-be-tested hydrogel for short), whose mechanical properties are tested. More specifically, this embodiment determines the gelation time and adhesion of each to-be-tested hydrogel. The gelation time is determined by measuring the time required, after the addition of tyrosinase, for a precursor compound put into a container and added with tyrosinase to turn into a dark brown gel that remains stuck to the bottom of the container when the container is placed upside down. Adhesion (or adhesive stress) is an important mechanical parameter of a material and is determined generally as follows. A collagen film is fixed on a surface portion of each of two aluminum plates, and the collagen-film-coated surfaces of the two aluminum plates are bonded together with a to-be-tested hydrogel (50 μL). The aluminum plates bonded with the to-be-tested hydrogel are then placed in a high-humidity environment (e.g., placed above the surface of a phosphate-buffered saline (PBS) solution if in a closed system) or completely submerged in the PBS solution. After 24 hours, the adhesive stress of the to-be-tested hydrogel is measured by the shearing test method (Instron MINI 44).

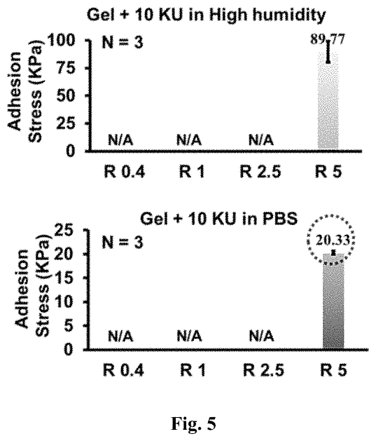

FIG. 4 shows gelation times corresponding to this embodiment, and FIG. 5 shows adhesive stress measurements corresponding to this embodiment. As shown in FIG. 4 and FIG. 5, the gelation time is shortened as the D.S. increases, and the adhesive stress measurements of group R5 are higher than those of the other groups (which have lower D.S. values) regardless of whether the to-be-tested hydrogels are placed in a high-humidity environment or completely submerged in a PBS solution. It can be known from the above that an increase of the D.S. not only accelerates gelation, but also allows the corresponding hydrogel product to have relatively high adhesion.

2-1-2 Analysis of Biocompatibility Corresponding to Different D.S. Values

As the present invention is intended to be applied to the field of biomaterials, this embodiment analyzes the correlation between the D.S. and bio compatibility.

The experimental steps of this embodiment are as follows. To begin with, mouse L929 cells (in Dulbecco's modified Eagle's medium (DMEM)-high glucose, with 10% fetal bovine serum (FBS) and 1% penicillin/streptomycin) are kept in an incubator (5% CO₂, 37° C.) and then seeded onto a 96-well plate (105 cells/ml) until confluence takes place so that the cells can be used in the experimental steps that follow. The precursor compound in each of groups R0.4, R1, R2.5, R5, and R6 (in which the D.S. value varies from one group to another) is used at a fixed concentration of 10 wt % and mixed with tyrosinase of a fixed concentration (10 KU) to form a to-be-tested hydrogel. Each to-be-tested hydrogel is then added with DMEM-high glucose (10% FBS, 1% penicillin/streptomycin/amphotericin (PSA)) and kept at 37° C. for 24 hours according to the ISO 10993 standard. The to-be-tested hydrogels are then used to culture the L929 cells so that the biocompatibility of the to-be-tested hydrogels can be determined.

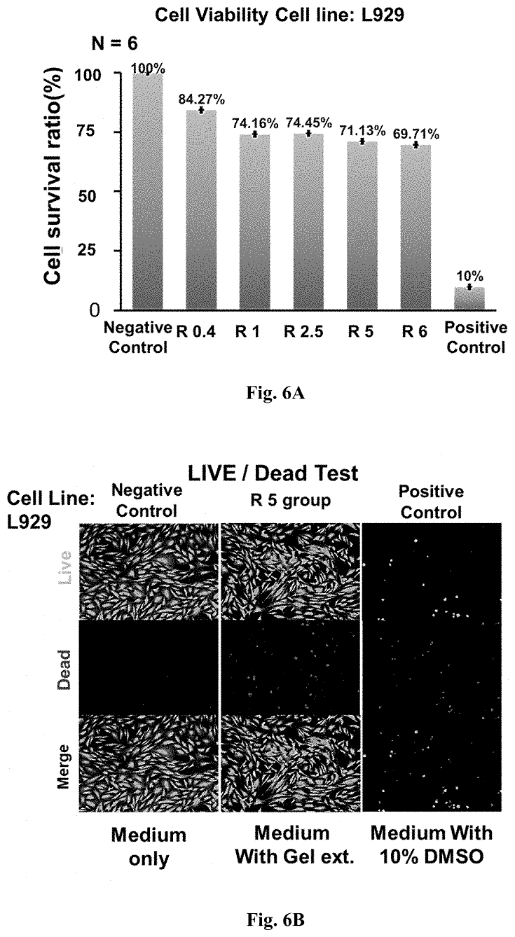

Here, biocompatibility is determined by an MTT assay and a LIVE/DEAD cell viability assay. An MTT assay is a test based on the property of the dye MTT (3-(4,5-dimethylthiazol-2-yl)-2,5-diphenyltetrazolium bromide) that it can be reduced and crystalized by a living cell; so, by measuring absorbance, the number of living cells in a sample can be assessed. A LIVE/DEAD cell viability assay is performed by observing the fluorescence performance of different dyes in living/dead cells so as to assess cell viability. FIG. 6A is a plot showing an MTT cell survival rate analysis result corresponding to this embodiment, and FIG. 6B shows a LIVE/DEAD cell viability assay result corresponding to this embodiment. The negative control groups and positive control groups shown in FIG. 6A and FIG. 6B are samples cultured in a common cell culture medium and in a cell culture medium with 10% dimethyl sulfoxide (DMSO), respectively. As can be seen in FIG. 6A, a rise of the D.S. does lower the survival rate of cells. Considering that a balance between the tensile strength and biocompatibility of a biomaterial is desirable, this embodiment further performs a LIVE/DEAD cell viability assay on group R5 in particular, and as shown in FIG. 6B, not only is the number of dead cells in the L929 cell sample that uses the to-be-tested hydrogel in group R5 as the culture medium rather small, but also there is no significant change in morphology of the living cells as compared with the living cells in the negative control group.

2-1-3 Correlation between the Concentration of Tyrosinase and the Gelation Time

This embodiment analyzes the correlation between the concentration of tyrosinase and the gelation time. The method of measuring the gelation time has been described above and therefore will not be repeated. In this embodiment, the R5 precursor compound, at 10 wt %, is reacted separately with tyrosinase of a concentration of 1, 2, 5, or 10 KU, and the gelation time measurements are plotted in FIG. 7, which shows that the concentration of tyrosinase is negatively correlated to the gelation time of a sample.

3. Addition of tyrosinase and calcium peroxide to cause gelation of the ALG-TYR precursor compound

This section aims to analyze the hydrogel produced by simultaneously adding tyrosinase and calcium peroxide to the ALG-TYR precursor compound to cause gelation thereof. The correlation between the variables involved and the properties of the hydrogel is also examined.

3-1 Spectrum Analysis

In this embodiment, the following four sample groups are tested by FTIR: ALG-TYR precursor compound, tyrosinase, 10 wt % ALG-TYR precursor compound+10 KU tyrosinase, and 10 wt % ALG-TYR precursor compound+10 KU tyrosinase+0.8 mM calcium peroxide.

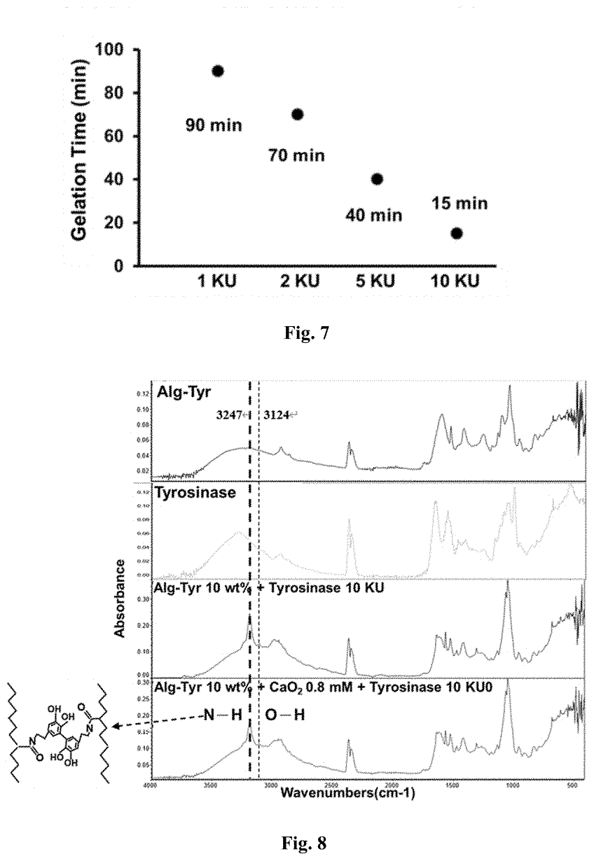

Referring to FIG. 8 for FTIR spectra corresponding to this embodiment, the 10 wt % ALG-TYR precursor compound+10 KU tyrosinase+0.8 mM calcium peroxide group shows noticeable peaks at 3247 and 3124 cm⁻¹, indicating the existence of NH and OH bonds. It can therefore be inferred that after tyrosinase and calcium peroxide are added to an ALG-TYR precursor compound, the tyrosinase catalyzes the formation of an NH bond on the ALG-TYR precursor compound and allows oxygen to be produced.

3-2 Morphological Analysis

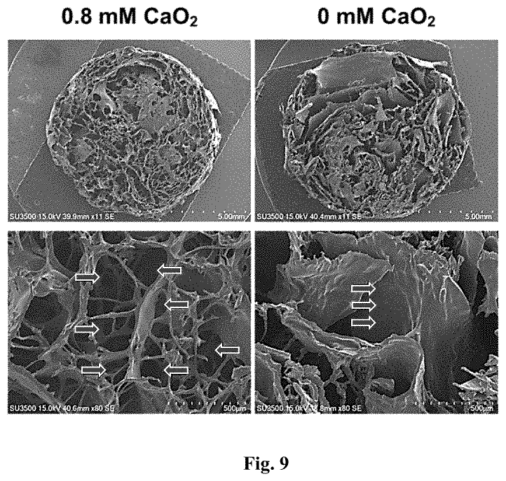

In this embodiment, scanning electron microscopy (SEM) is used to analyze the effect of the addition, or no addition, of 0.8 mM calcium peroxide on the enzymatic gelation of ALG-TYR. The resulting SEM images are shown in FIG. 9, in which it can be seen that the group added with 0.8 mM calcium peroxide shows a denser and better-organized structure with more inter-connection pores (indicated by the arrows) than the group to which no calcium peroxide is added. It can therefore be inferred that calcium peroxide contributes to the formation of sufficient crosslinking points and pore space and hence to the provision of inter-connection pores and high porosity. The finding of this morphological change is beneficial to the modification, attachment, migration, proliferation, and potential differentiation of innate cells.

3-3 Analysis of Mechanical Properties

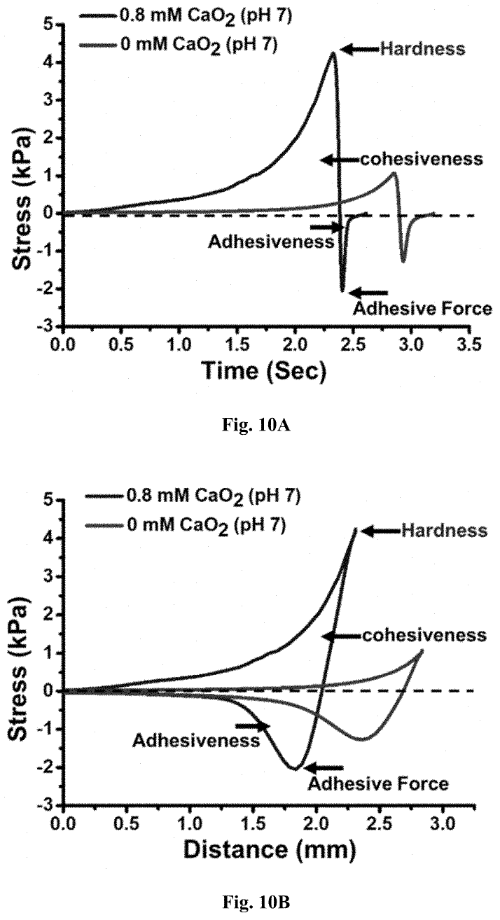

In this embodiment, a texture profile analysis (TPA) is performed to analyze the effect of adding 0.8 mM calcium peroxide to an ALG-TYR precursor compound (10 wt %)+tyrosinase (10 KU) in a pH 7 environment on the hardness, cohesion, and adhesion of the resulting hydrogel. A TPA can be used in different modes as needed in order to test the mechanical properties of a material. More specifically, the steps of the TPA performed in this embodiment include preparing a cylindrical hydrogel (with a diameter of 8 mm and a height of about 8 mm) with a mold, placing the hydrogel in a humid environment for at least 48 hours after the hydrogel completely gels, and then analyzing the hydrogel with a texture analyzer (TA.XT Plus Texture Analyzer). FIG. 10A is a plot showing a stress-time relationship corresponding to this embodiment, and FIG. 10B is a plot showing a stress-distance relationship corresponding to this embodiment. It can be seen in FIG. 10A and FIG. 10B that the hydrogel produced by adding 0.8 mM calcium peroxide exhibits notably higher hardness, cohesion, and adhesion than the hydrogel to which no calcium peroxide is added.

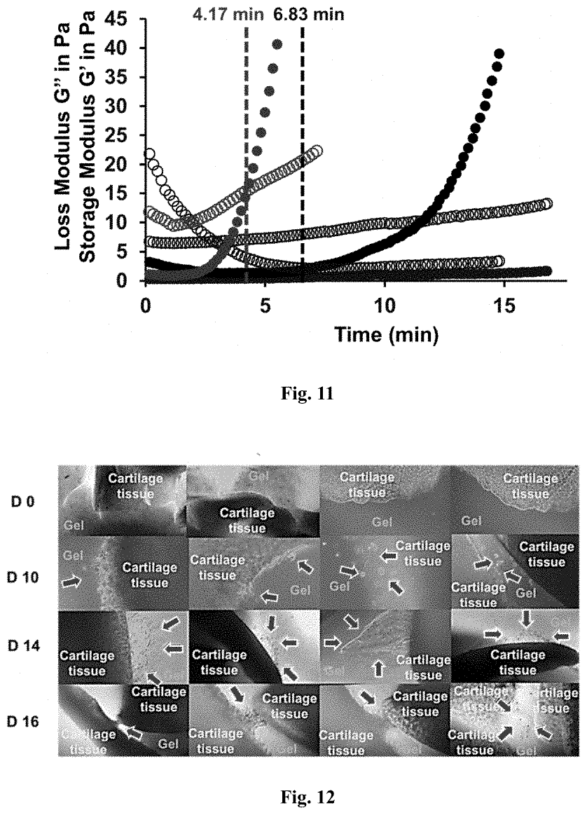

This embodiment further uses a rheometer (MCR 302, Anton-Paar) to analyze the effect of adding 0.8 mM calcium peroxide to an ALG-TYR precursor compound (10 wt %)+tyrosinase (10 KU) on the rheological properties of the resulting hydrogel. FIG. 11 shows rheological property test results corresponding to this embodiment, with the light grey color representing grouop-R5 precursor compound+tyrosinase+calcium peroxide, the black color representing group-R5 precursor compound+tyrosinase, and the dark grey color representing group-R5 precursor compound alone. The solid circles in FIG. 11 represent viscosity and correspond to the coordinate axis for the storage modulus G′. The hollow circles, on the other hand, represent elasticity and correspond to the coordinate axis for the loss modulus G″. It can be seen in FIG. 11 that the group-R5 precursor compound+tyrosinase+calcium peroxide group has a relatively high gelation speed, and that the storage modulus G′ line and loss modulus G″ line of the group-R5 precursor compound+tyrosinase+calcium peroxide group intersect, indicating successful gelation.

3-4 In Vitro Experiment and Analysis

This embodiment performs an in vitro experiment whose steps are generally as follows. Fresh porcine cartilage is obtained from a slaughterhouse and sterilized with betadine, which is alcohol-free. The porcine articular cartilage is cut into fragments (smaller than 5 mm²) with a surgery blade, and the fragments obtained are rendered into even smaller fragments (smaller than 1 mm²) by a homogenizer having a particular mesh size (Reveille™). The smaller articular cartilage fragments obtained are then treated with an enzyme (Liberase, reconstituted with 10 ml Hanks' balanced salt solution (HBSS) per vial) for 20 min in order to remove the matrix. After that, 0.2 ml of group-RS precursor compound is added with tyrosinase (10 KU) and 0.8 mM calcium peroxide in order to gel enzymatically and thereby form a to-be-tested hydrogel. To examine the bio-interaction between the cartilage and the to-be-tested hydrogel, the hydrogel is placed on the cover slip of a confocal dish, and the treated smaller cartilage fragments are blended into the hydrogel on the cover slip of the confocal dish to facilitate observation through an optical microscope and further analysis.

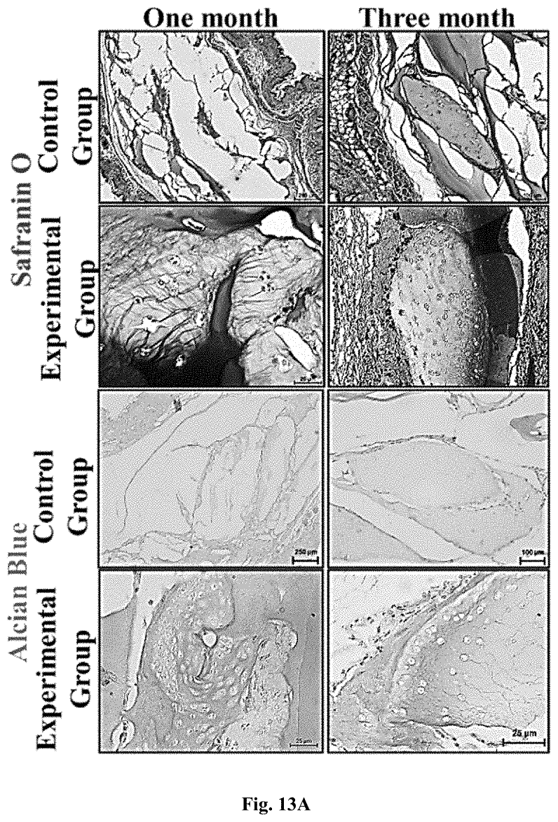

Referring to FIG. 12 for optical microscope images corresponding to this embodiment, the boundary between the cartilage and the hydrogel is clearly visible under the optical microscope on day 0. Observation on day 10 reveals that a small number of cells have migrated from the cartilage to the hydrogel. Further observation on day 14 to 16 reveals that a certain amount of extracellular matrix (ECM) has gradually formed on the cartilage interface, and that two cartilage fragments have fused together. It can be known from the above that the hydrogel contributes to the proliferation and migration of chondrocytes and the formation of ECM.

3-5-1 In Vivo Experiment and Analysis 1

In this embodiment, the control group (a mixture of ALG+calcium chloride and porcine cartilage fragments) and the experimental group (a mixture of ALG-TYR precursor compound+tyrosinase+calcium peroxide and porcine cartilage fragments) are separately implanted into subcutaneous pockets of nude mice. The pockets are cut open after one or three months in order to carry out microscopic histological examination and immunostaining, thereby assessing the in vivo biostability of the control group and of the experimental group and the interaction between the materials. More specifically, the stains used in this embodiment are safranin 0, Alcian blue, and hematoxylin and eosin (H&E).

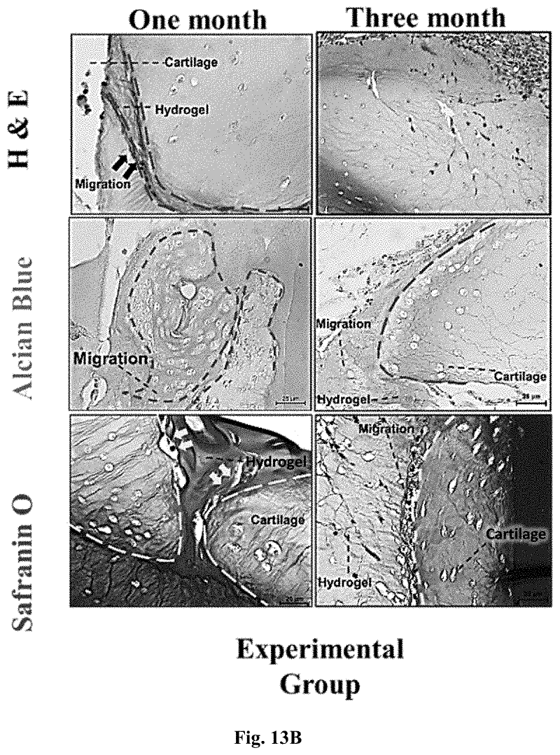

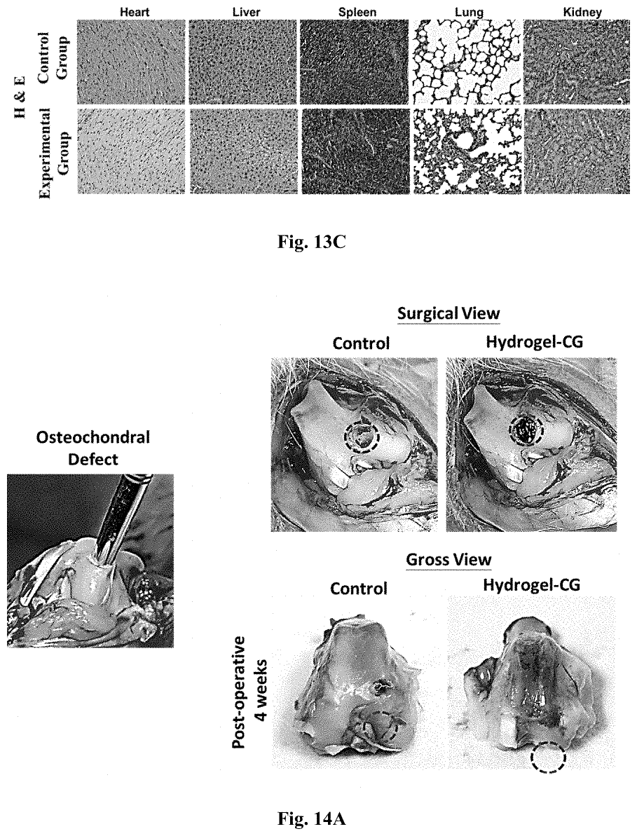

FIG. 13A shows immunohistochemical analysis results of the experimental group and control group in this embodiment, FIG. 13B shows more immunohistochemical analysis results of the experimental group in this embodiment, and FIG. 13C shows immunohistochemical analysis results of certain soft tissues adjacent to the implanted experimental group/control group in this embodiment. It can be seen in FIG. 13A to FIG. 13C that there is more chondrocyte extracellular matrix deposition around the cartilage fragments in the experimental group than in the control group; that the experimental group preserves more cartilage fragments than the control group and allows the gaps between the cartilage fragments to close up, the cartilage fragments to integrate with one another, and cells to migrate from the cartilage fragments into the hydrogel; and that visible toxicity of the implanted hydrogel (e.g., hyperemia, edema, or necrosis) is not found in the neighboring organs or soft tissues (including the heart, liver, spleen, lungs, and kidney) of the experimental animals.

3-5-2 In Vivo Experiment and Analysis 2

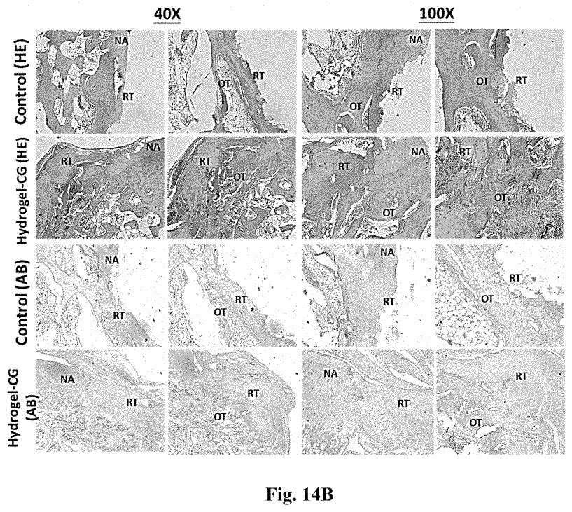

In this embodiment, the control group (a mixture of ALG+calcium chloride and porcine cartilage fragments) and the experimental group (a mixture of ALG-TYR precursor compound+tyrosinase+calcium peroxide and porcine cartilage fragments) are separately implanted into defective cartilage portions of rabbits. The implanted areas are cut open after one month in order to carry out observation with the naked eye, microscopic histological examination, and immunostaining, with a view to assessing the progress of tissue repair. The examination results are shown in FIG. 14A and FIG. 14B. The stains used in this embodiment are Alcian blue and H&E.

In terms of appearance (see FIG. 14A), the condylar surface of the joint implanted with the control group is recessed and uneven, indicating that the tissue has been repaired to a very limited degree. By contrast, the defect implanted with the hydrogel-cartilage group is smaller and shallower than that implanted with the control group and is filled with a large amount of repair tissue.

In addition, each microscopic histological image is divided into a natural cartilage area (NA), a repair area (RT), and a subchondral bone area (OT) to facilitate observation and assessment of the repair results. As shown in FIG. 14B, the interface between the undamaged natural cartilage and repair area corresponding to the control group is uneven. The defect implanted with the hydrogel-cartilage group, however, is filled with growable implanted cartilage fragments, with the implanted cartilage, the natural cartilage, and the underlying subchondral bone sufficiently integrated in the lateral direction. Besides, the repair area corresponding to only the hydrogel-cartilage group shows positive Alcian blue staining, which indicates the deposition of chondrocyte extracellular matrix.

3-6 Analysis of the Optimal Concentration of Calcium Peroxide

In this embodiment, the effect of adding calcium peroxide at different concentrations on mechanical properties and biocompatibility is analyzed.

3-6-1 Correlation between Different Calcium Peroxide Concentrations and the Mechanical Properties of Corresponding Hydrogels

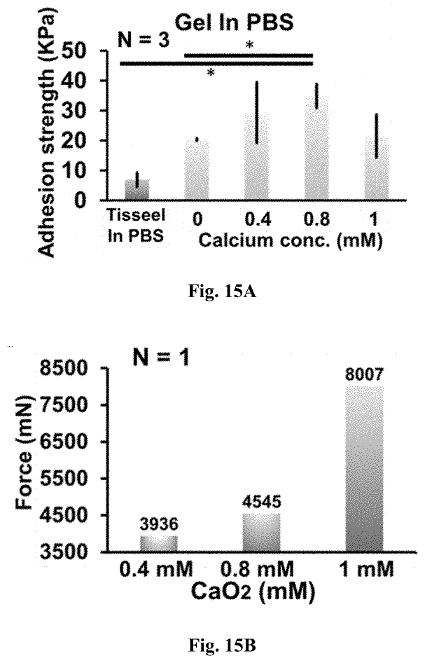

In this embodiment, the adhesion and tensile strength of hydrogels produced respectively by adding calcium peroxide at different concentrations to group-R5 precursor compound+10 KU tyrosinase submerged completely in a PBS solution are tested. More specifically, the tensile strength in question includes adhesion, cohesion, and compression. FIG. 15A and FIG. 15B are plots showing hydrogel adhesion analysis results and overall hydrogel tensile strength analysis results corresponding to this embodiment. As shown in FIG. 15A, the group whose calcium peroxide concentration is 0.8 mM has the highest adhesion, and adhesion is lowered rather than increased when the calcium peroxide concentration is further raised to 1.0 mM. Furthermore, it can be seen in FIG. 15B that the group whose calcium peroxide concentration is 1.0 mM has the highest tensile strength. It can therefore be inferred that the adhesion of a hydrogel is compensated by cohesion or compression when the calcium peroxide concentration exceeds 0.8 mM.

3-6-2 Correlation between Different Calcium Peroxide Concentrations and the Biocompatibility of Corresponding Hydrogels

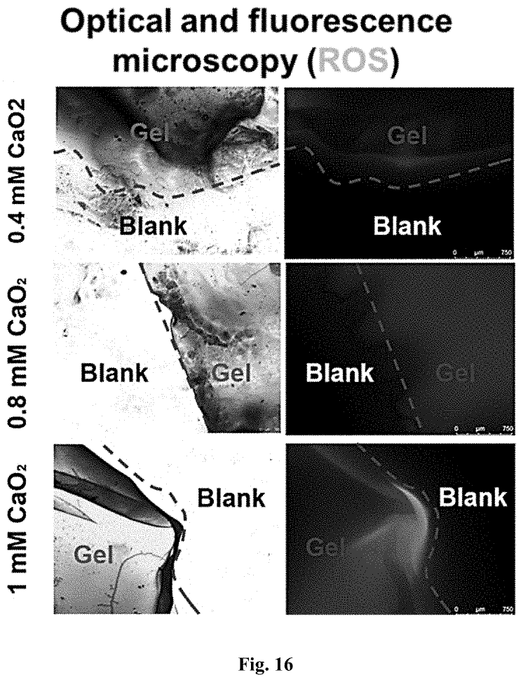

The states of the hydrogels produced respectively by adding calcium peroxide at different concentrations to group-R5 precursor compound+10 KU tyrosinase are further observed through an optical microscope and a fluorescence microscope. Referring to FIG. 16 for optical/fluorescence microscope images corresponding to this embodiment, oxidation products are significantly increased when the calcium peroxide concentration reaches 1 mM.

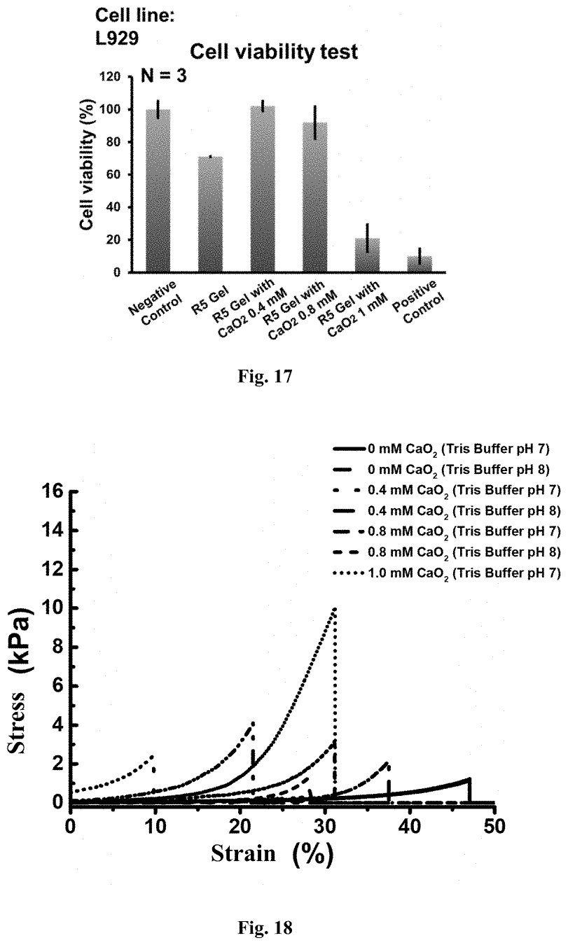

Besides, the effect of the concentration at which calcium peroxide is added on cell viability is analyzed by an MTT assay (whose experimental details have been stated above and therefore will not be repeated). Referring to FIG. 17 for a plot showing the MTT cell viability assay result corresponding to this embodiment, the groups whose calcium peroxide concentrations are 0.4 or 0.8 mM exhibit higher cell viability than the other groups. Therefore, considering all the aspects discussed above, the concentration at which calcium peroxide is added is preferably between 0.4 and 1 mM, and more preferably between 0.4 and 0.8 mM. More specifically, the calcium peroxide concentration may be 0.4, 0.5, 0.6, 0.7, or 0.8 mM.

3-7 Analysis of Reaction Conditions

In this embodiment, the effects of different buffer solutions and different pH values on the mechanical properties of corresponding hydrogel products are analyzed.

It is well known in the art that a Tris, or tris(hydroxymethyl)aminomethane, buffer solution is less likely to precipitate calcium salts and can better preserve the solubility of metal salts than normal saline and a phosphate-buffered saline, and that the pH value may affect the enzyme activity of tyrosinase and the release of oxygen from calcium peroxide. Therefore, the gelation of ALG-TYR precursor compounds in different pH conditions and in different buffer solutions (to which calcium peroxide and tyrosinase are added) is further analyzed.

More specifically, this embodiment performs a TPA (whose details have been given above and therefore will not be repeated) in conjunction with a compression test and determination of the Young's modulus in order to find the optimal buffer solution and optimal ambient pH value. FIG. 18 is a plot showing a stress-strain relationship corresponding to this embodiment, and Table 2 is a list of Young's moduli corresponding to this embodiment. As shown in FIG. 18 and Table 2, when the calcium peroxide concentration (0.8 mM) remains the same, the group using a pH value of 7 and a saline buffer solution and the group using a pH value of 8 and a Tris buffer solution have lower mechanical strength than the group using a pH value of 7 and a Tris buffer solution. This may be attributable to the fact that tyrosinase is more active in a pH 7 environment than at other pH values, and that a Tris buffer solution can prevent metal precipitation and increase the solubility of metal-oxide-based salts, thereby enhancing the reactivity of calcium peroxide and hence the provision of sufficient soluble calcium ions and oxygen. Considering all the aspects discussed above, preferred reaction conditions entail a pH value of 7 and a Tris buffer solution.

TABLE 2

| | Young's |
|---|---|
| Groups | modulus |
| calcium peroxide 0 mM/Tris/pH = 7 | 0.20 |
| calcium peroxide 0.4 mM/Tris/pH = 7 | 0.40 |
| calcium peroxide 0.8 mM/Tris/pH = 7 | 0.69 |
| calcium peroxide 0.8 mM/saline/pH = 7 | 0.09 |
| calcium peroxide 0.8 mM/Tris/pH = 8 | 0.53 |
| calcium peroxide 1.0 mM/Tris/pH = 7 | 1.69 |

In conclusion, the hydrogel composition disclosed herein, the method disclosed herein for manufacturing the hydrogel composition, and the enzymatically formed hydrogel composition disclosed herein provide not only high biocompatibility, but also desirable mechanical properties and a high gelation speed.

What is claimed is:

1. A hydrogel composition, comprising:
a plurality of polymers, wherein each of the polymers includes a backbone, and the backbone includes a plurality of carboxyl groups and a branch formed by tyramine,
wherein any two of the polymers have a bond between adjacent branches, and at least one of the plurality of carboxyl groups is chelated with a calcium ion.

2. The hydrogel composition of claim 1, wherein the backbone is selected from the group consisting of gelatin, chitosan, heparin, cellulose, dextran, dextran sulfate, chondroitin sulfate, keratan sulfate, dermatan sulfate, alginate, collagen, albumin, fibronectin, laminin, elastin, vitronectin, hyaluronic acid, fibrinogen, a multi-arm polymer and a combination thereof

3. The hydrogel composition of claim 1, wherein the backbone is a polysaccharide copolymer or a polysaccharide homopolymer.

4. The hydrogel composition of claim 1, wherein the bond is an enzymatic oxidative coupling.

5. The hydrogel composition of claim 4, wherein the enzymatic oxidative coupling is formed by in situ cross-linking of the polymers in an environment with an enzyme and calcium peroxide, wherein the concentration of calcium peroxide is between 0.4 and 1mM, and the enzyme is tyrosinase or horseradish peroxidase.

6. An enzymatically formed hydrogel composition as represented by formula (II), which includes:
a plurality of polymers, each of which comprises a homogenous or heterogeneous backbone with a structure as represented by formula (I);
wherein at least one carboxyl group of the backbone and at least one calcium ion are chelated into a structure of formula (II),

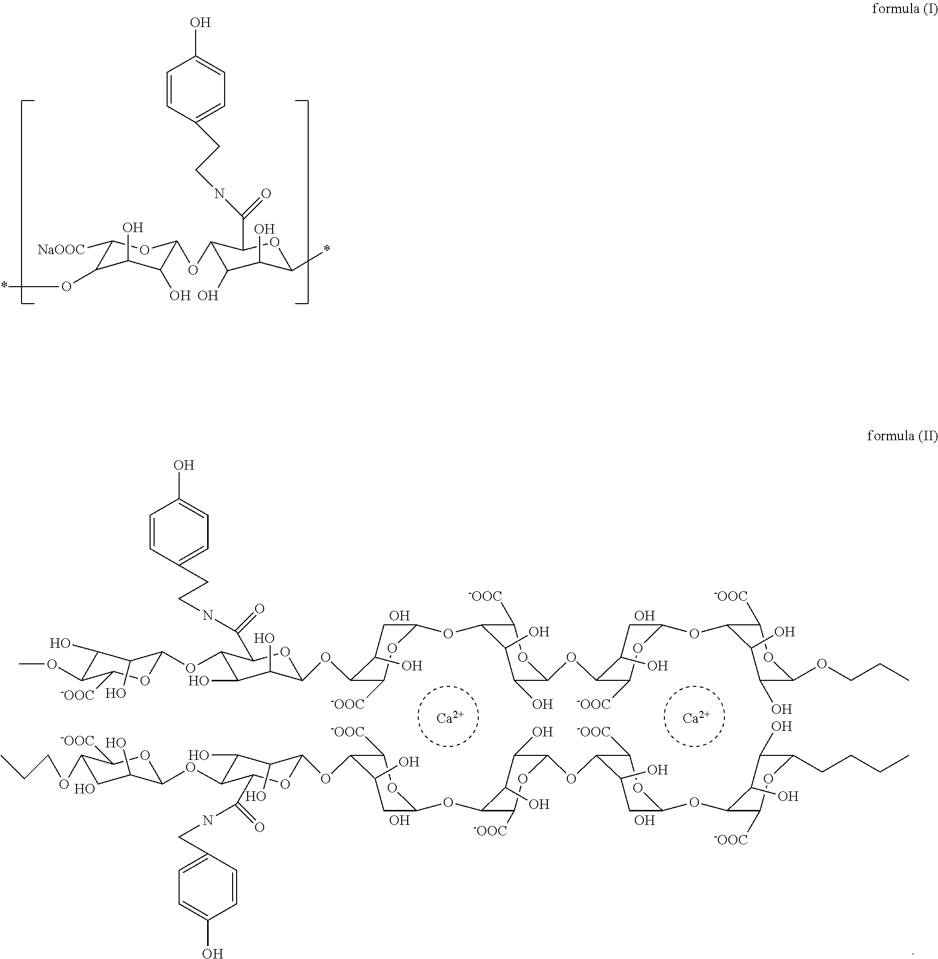

7. The enzymatically formed hydrogel composition of claim 6, wherein the backbone includes a branch formed by tyramine, and adjacent branches on any two of the polymers have a bond as shown in the structure of formula (III):

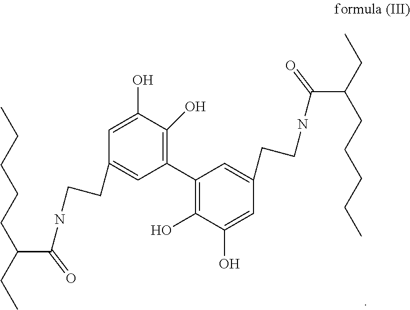 [SMILES: CCCCCC(CC)C(=O)NCCc1cc(O)c(O)c(-c2cc(CCNC(=O)C(CC)CCCCC)cc(O)c2O)c1]

8. The enzymatically formed hydrogel composition of claim 7, wherein the structure of formula (II) and the bond of the structure of formula (III) are formed by in-situ cross-linking of the polymers in an environment with an oxidase and calcium peroxide.

9. The enzymatically formed hydrogel composition of claim 8, wherein the oxidase is tyrosinase or horseradish peroxidase.

10. The enzymatically formed hydrogel composition of claim 8, wherein the concentration of calcium peroxide is between 0.4 and 1mM.

11. A method for manufacturing a hydrogel composition, which includes the following steps:
(a) reacting tyramine with a backbone of a polymer to produce a precursor polymer; and
(b) adding a cross-linking accelerator and an ion chelating agent to cross-link a plurality of the precursor polymers to form a hydrogel composition.

12. The method of claim 11, wherein in step (a), the reaction ratio of the polymer and tyramine is between 1:0.4 and 1:6.

13. The method of claim 11, wherein the cross-linking accelerator is tyrosinase or horseradish peroxidase.

14. The method of claim 11, wherein the ion chelating agent is calcium peroxide.

15. The method of claim 11, wherein in step (b), the reaction concentration of the ion chelating agent is between 0.4 and 1 mM.
